(19) United States
(12) Patent Application Publication
(10) Pub. No.: US 2025/0237008 A1
(43) Pub. Date: Jul. 24, 2025

(54) METHOD AND APPARATUS FOR MAKING AN ARTICLE FROM FILAMENTS CONTAINING BACTERIOPHAGES

(71) Applicant: OXFORD SILK PHAGE TECHNOLOGIES LTD, Oxford (GB)

(72) Inventors: Laure MAGNAN, Oxford (GB); Stephanie LESAGE, Oxford (GB)

(21) Appl. No.: 18/836,596

(22) PCT Filed: Feb. 8, 2023
(86) PCT No.: PCT/GB2023/050286

§ 371 Date: Aug. 7, 2024

(30) Foreign Application Priority Data
Feb. 8, 2022 (GB) 2201607.5

(51) Int. Cl. D06M 16/00; A61L 17/00; D06B 1/02; D06B 21/00; D06M 10/02; D06M 23/16; D06M 101/12

(52) U.S. Cl. CPC D06M 16/003; A61L 17/005; D06B 1/02; D06B 21/00; D06M 10/02; D06M 23/16; D06M 2101/12; D10B 2211/22; D10B 2401/022; D10B 2509/00

(57) ABSTRACT

The present invention provides a method of manufacturing an article (10) comprising one or more filaments (12) of material having a hydrophilic surface (12a) comprising the steps of: forming a first solution (16) containing one or more bacteriophages (18a) in suspension in a carrier fluid (20) and depositing them upon one or more filament (12), in which said deposition of said first solution (16) is by dispensing drops (22) thereof from a drop dispenser (24) and by the step of depositing a pre-determined amount of said first solution per unit length on said first filament (12) and then forming said one or more first filaments (12) into a final article (10).

### FIELD OF THE INVENTION

The present invention relates to a method of manufacturing an article having bacteriophages attached thereto and relates particularly but not exclusively to a method of manufacturing an article comprising or formed of a filamentous material having bacteriophages deposited thereon. It also relates to an article made in accordance with the method and an article comprising one or more filaments of material having bacteriophages contained thereon or therein. Other elements of the present invention include an apparatus for manufacturing an article, an article manufactured by the method described herein and an apparatus and method of aligning bacteriophages on a filament.

### BACKGROUND TO THE INVENTION

Resistance to conventional antibiotics has become increasingly prevalent and threatens to be human health's next biggest challenge, expected to kill 10 m per year and increase costs to the health care systems annually from 2050. One promising alternative method to prevent and treat bacterial infections is the use of bacteriophages, natural viruses, and enemies of bacteria.

One challenge with bacteriophage therapy is the instability of the bacteriophages when stored in solution and their rapid elimination from the human body when administered as an alternative treatment to antibiotics. Cocktails of bacteriophages are particularly unstable as highlighted in a 2015 literature publication summarising Europe's first largest bacteriophage treatment clinical trial.

Stabilising bacteriophages by covalently attaching them onto substrate materials is known and may comprise the step of covalently attaching bacteriophages to surfaces by activating surfaces with corona or using coupling agents, such as described in first patent EP1496919. It is also known to protect bacteriophages during storage, by treating them with glutaraldehyde of trealose.

Whilst the patent claims to allow head-group specific binding, the method described only enables the random attachment of bacteriophages to a surface and does not prevent the horizontal alignment of the tails to the surface or propose a method to avoid the horizontal alignment of the tails to the surface. Bacteriophages bind to bacteria through their tails and the horizontal alignment of bacteriophages on the surface may reduce the infectivity of the bacteriophages and thus their effectiveness against pathogens.

Whilst the patent also claims to enable the bonding of a plurality of strain specific bacteriophages to the surfaces, the method only enables the random addition of a solution containing a mix of different bacteriophage strains. Unfortunately, it is well known that cocktails of bacteriophages in a solution may interfere with one another thereby potentially reducing the overall infectivity when added altogether so this approach may not be as effective as might be desired.

Another patent entitled “Production of immobilised bacteriophages” (EP18207889) teaches pressure spraying of micro-droplets containing bacteriophages onto fine particles or filaments or planar surfaces. Shortcomings of this method include the inability to deposit exact concentrations of bacteriophages onto each particle/filament, as the pressure spraying method leads to the random dissemination of the micro droplets onto the powder, as well as the inability to efficiently deposit high concentrations of bacteriophages onto each particle due to the small volumes involved.

Whilst this second patent suggests a more equal spreading of the bacteriophages onto particles, it does not ensure that every particle or substrate received a very precise concentration of bacteriophages and does not ensure that no bacteriophage has been wasted by missing its target substrate when disseminated onto the surface.

In this second patent, an electric field is applied to both the material and the bacteriophage-containing solution, to promote the attachment of the phage capsids to the surface of the materials. However, a negative electric charge has never been applied around the surface of the material after bacteriophage deposition to attract the positively charged tails in most optimal position. Therefore, not all bacteriophages will have the active tail facing outwards away from the attachment surface.

Other methods described in the literature involve the dipping of materials in a solution containing bacteriophages to coat the material with bacteriophages present in the solution. This method however cannot ensure exact quantities of bacteriophages are transferred between the solution and the material, resulting in a random deposition of bacteriophages on random areas of the material, and in a high percentage waste of bacteriophages in the solution.

In production of products that use bacteriophages the cost of the bacteriophage used to create the product makes up a large portion of the cost of the product. Poor alignment of bacteriophages reduces the effectiveness of the bacteriophages against pathogens requiring more bacteriophage. Existing production methods give poor control of bacteriophage concentration and volumes used and result in wastage of expensive bacteriophage solution.

It is an object of the present invention to obviate and/or mitigate at least some of the above disadvantages. A more specific object of the invention is to provide alternative methods for the manufacture of articles consisting of or formed of filaments of material comprising bacteriophages, where for the first time very precise concentrations of bacteriophages are accurately immobilised onto determined surface areas of a substrate, reducing and possibly eliminating waste of the bacteriophage solution and enabling the addition of patterns of multiple strains of bacteriophages onto a same or different zones and/or regions of the article being formed. This also has the advantage of enabling the addition, on a same device, of multiple cocktails of synergetic phages against a same bacteria strain, whilst maintaining phages of antagonistic effect separate, or of multiple cocktails targeting different bacteria, Other objects of the present invention include but are not limited to providing an apparatus suitable for use with the method of the present invention and an article produced by the method of the present invention. A still further aspect of the present invention provide a method and apparatus for aligning bacteriophage relative to a filament such as to ensure the tails of the bacteriophages are aligned pointing away from the filament. The present invention also makes it possible to produce a filament that can be stored for later assembly with other filaments containing the same or different bacteriophages into a finished article. Desirably, such an article could be produced with a bespoke selection of specific bacteriophages selected to suit the particular medical application of the article. The invention, therefore, makes it possible to produce an off-the-shelf designer product or a bespoke product.

Experiments included herein show that excess bacteriophage solution deposited at the same location or overlapping locations can result in reduced effectiveness of the finished article even if the solution contains the same bacteriophage. The reduction in efficacy may be further compounded if the drops are of solutions containing different bacteriophages with antagonistic effects.

### STATEMENT OF INVENTION

According to one aspect of the present invention, there is provided a method of manufacturing an article comprising one or more filaments of material having a hydrophilic surface comprising the steps of: forming a first solution containing one or more bacteriophages in suspension in a carrier fluid; taking one or more first filaments; and depositing upon said one or more first filaments said first solution containing said one or more bacteriophages characterised in that said deposition of said first solution is by dispensing drops thereof from a drop dispenser and by the step of depositing a pre-determined amount of said first solution per unit length on said first filament and forming said one or more first filaments into a final article.

There may be provided a step of forming a second solution containing one or more second bacteriophages different from said first bacteriophages in suspension in a second carrier fluid; taking one or more second filaments; and depositing upon said one or more second filaments said second solution containing said one or more second bacteriophages a pre-determined amount of said second solution per unit length on said second filament and forming said one or more first filaments into a final article along with said second filaments.

Optionally, the one or more first filaments each having a first region discrete from a second region. The method optionally including the further steps of forming a second solution containing one or more second bacteriophages different from said first bacteriophages in suspension in a second carrier fluid; depositing upon said first region a pre-determined amount per unit length of said first bacteriophage solution; and depositing upon said second region a pre-determined amount per unit length of said second bacteriophage solution; and forming said one or more filaments (12) into a final article (10).

The forming step may comprise one or other of: weaving, braiding, plating, knitting, embroidering an article or forming a non-woven article.

Preferably, said bacteriophages are mixed in said solution at a concentration of bacteriophages per unit solution of between 10² PFU/mL to 10¹² PFU/mL. Still more preferably the bacteriophages are mixed in said solution at a concentration of bacteriophages per unit solution of between 10⁵ PFU/mL to 10⁹ PFU/mL.

Advantageously, said filament has a length L and the method includes the step of depositing said solution at discrete separated positions along the length L of said filament.

The method may include the step of simultaneously depositing multiple drops of said solution onto said filament at discrete separated positions along said filament. Preferably, with each of said multiple drops at discrete separated positions along said filament. Thereby allowing accurate saturation of the filament and/or separation of different phage solutions.

The method may include the step of depositing said drops on said filament at a spacing sufficient to saturate the entire length L of said filament or substantially the entire length of the filament.

There may be included the step of depositing said drops on said filament at a spacing insufficient to saturate the entire length of said filament, thereby to form discrete lengths of said filament without bacteriophages applied thereto.

A drop of a certain volume V can be easily shown to saturate a filament of a certain diameter for a length S by experimentation, for example by using a died solution to highlight the length of filament that is saturated which may be measured.

The method may include the step of manufacturing an article having two or more zones and forming said article of one or more filaments having different bacteriophages, thereby to form an article having different bacteriophages at different zones thereof.

The article may have an end portion and a non-end portion and the method may further include forming said article having a first filament having a first bacteriophage at said end portion and a second filament having a second bacteriophage at said non-end portion.

The method may include the step of mixing said bacteriophage containing solution prior to deposition on said filament.

The method may also include the step of modifying the surface of said filament to increase the surface hydrophilicity thereof. The step of modifying the surface of the filament may be by passing said filament through a plasma discharge.

In a particularly advantageous arrangement, there is included the step of passing the filament along electrode and applying an electric potential to align the bacteriophages relative to said filament. This may be done by passing the filament through a tubular electrode. Such an electrode may be cylindrical, square, triangular in cross-section or any other shape so long as it is able to create an electrical field potential such as to align the bacteriophages as described herein. It may also be possible to pass the filament between separated electrodes.

In one arrangement the first solution is deposited by simultaneously dispensing a plurality of drops thereof onto said filament from spaced apart drop dispensers.

According to a further aspect of the present invention, there is provided a method of manufacturing a filament comprising the steps of: forming a first solution containing one or more bacteriophages in suspension in a carrier fluid; taking one or more filaments; and depositing upon said one or more first filaments said first solution containing said one or more bacteriophages, characterised in that said deposition of said first solution is by dispensing drops thereof from a drop dispenser and by the step of depositing a pre-determined amount of said first solution per unit length on said first filament.

The method of the second aspect may include the step of mixing said bacteriophages in said solution at a concentration of bacteriophages per unit solution of between 10² PFU/mL to 10¹² PFU/mL or at a preferred concentration of bacteriophages per unit solution of between 10⁵ PFU/mL to 10⁹ PFU/mL.

The filament may have a length L and including the method may include the step of depositing said solution at discrete separated positions along the length L of said filament.

The method may include simultaneously depositing multiple drops of said solution onto said filament at discrete separated positions along said filament.

The method may also include the step of depositing said drops on said filament at a spacing sufficient to saturate the entire length L of said filament. Alternatively, the method may include the step of depositing said drops on said filament at a spacing insufficient to saturate the entire length L of said filament, thereby to form discrete lengths of said filament without bacteriophages applied thereto.

Advantageously, the method includes the step of mixing said bacteriophage containing solution prior to deposition on said filament.

For some materials, there me be included the step of modifying the surface of said filament to increase the surface hydrophilicity thereof. The modifying the surface of the filament may be by passing said filament through a plasma discharge.

In a particularly advantageous arrangement, there is included the step of passing the filament along electrode and applying an electric potential to align the bacteriophages relative to said filament. This may be done by passing the filament through a tubular electrode. Such an electrode may be cylindrical, square, triangular in cross-section or any other shape so long as it is able to create an electrical field potential such as to align the bacteriophages as described herein. It may also be possible to pass the filament between separated electrodes.

Preferably, said drops are dispensed having a size greater than 0.1 μL. Further preferably the drops are dispensed having a size range of between 0.2 μL and 100 μL.

According to a still further aspect of the present invention, there is provided an article comprising one or more filaments of material having a hydrophilic surface and containing within said hydrophilic surface one or more bacteriophages.

The present invention also describes a method of ensuring the bacteriophage tails are oriented perpendicular to the surface of the substrate, to increase infectivity, by using electric charge around the material once bacteriophages have been deposited.

Aspects of the present invention will now be more particularly described by way of example and with reference to the accompanying drawings, in which:

FIG. 1 is a diagrammatic view of a woven structure having filaments incorporating the present invention;

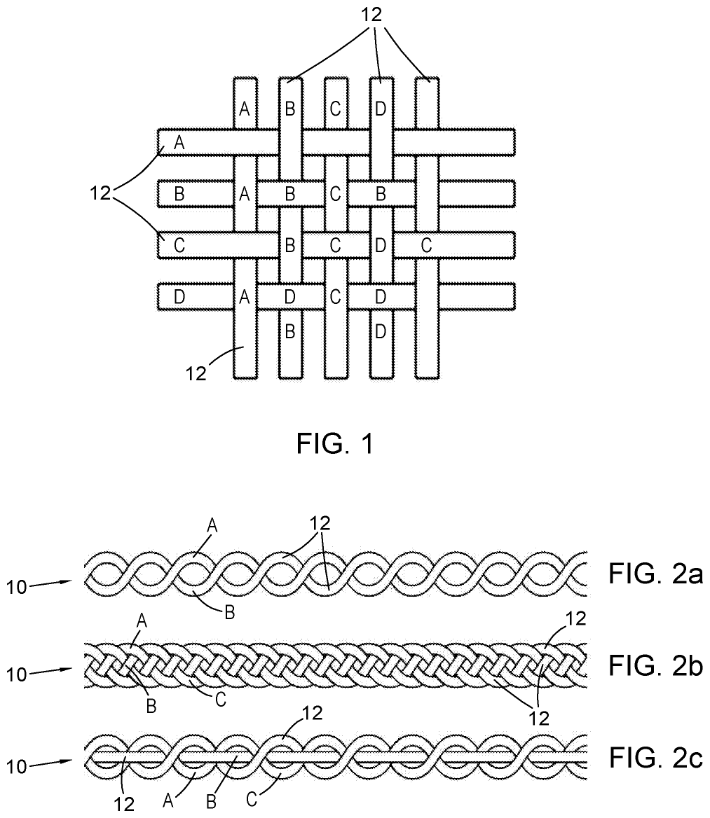

FIGS. 22 to 2b are examples of alternative braided structures of filaments having the present invention applied thereto;

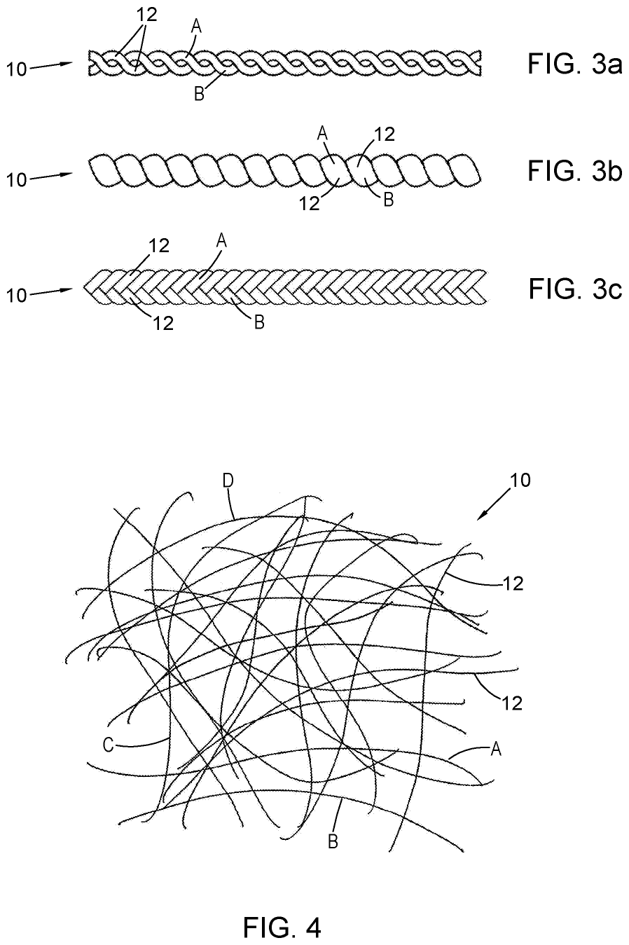

FIGS. 3a to 3c are still further examples of woven, twisted and/or braided structures to which the present invention has been applied;

FIG. 4 is an example of a non-woven structure to which the present invention has been applied and comprises a wadding of randomly distributed filaments to which the present inventive process has been applied;

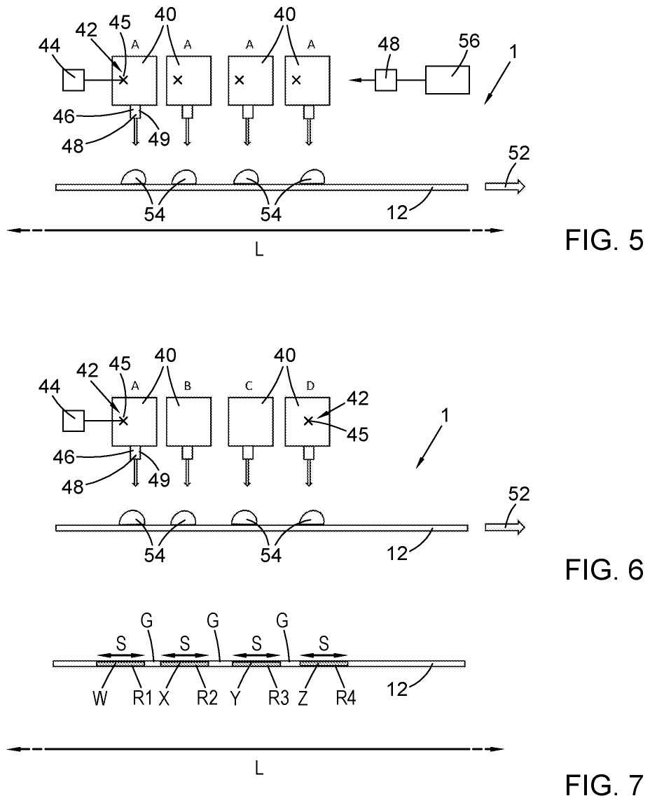

FIG. 5 is a schematic view of a first drop-dispensing arrangement;

FIG. 6 is an alternative schematic view of a drop dispensing arrangement;

FIG. 7 is a view of a filament after receiving multiple drops;

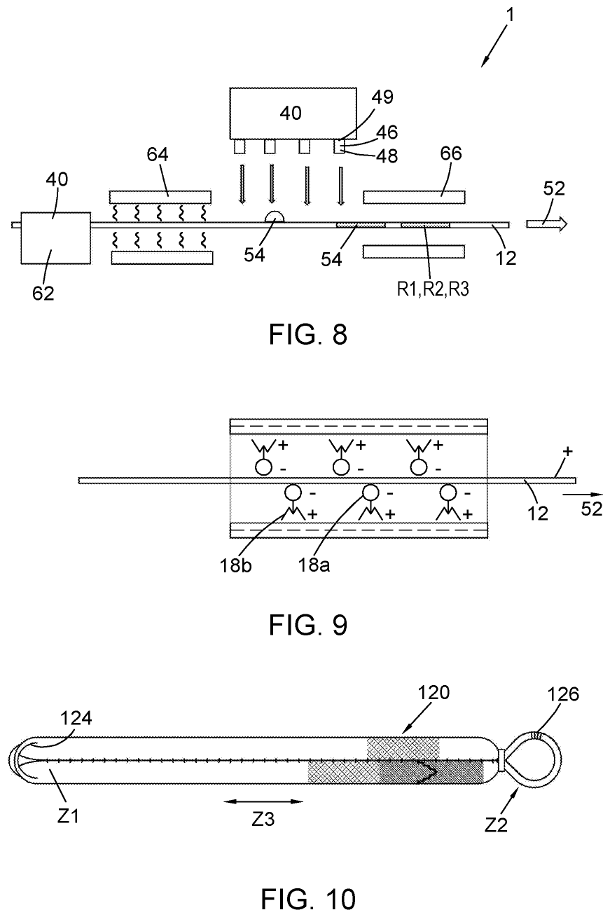

FIG. 8 is a diagrammatic representation of an arrangement of apparatus suitable for use in the production of a filament according to the present invention;

FIG. 9 is an expanded view of an arrangement used to align the bacteriophages on the filament; and

FIG. 10 is a representation of a sample final product which can be made form a filament subjected to the method of the present invention.

Referring now to the drawings in general but more particularly to FIGS. 1 to 4, an article or product 10 of the present invention comprises one or more filaments 12 of material having a hydrophilic surface 12a which may be natural to the material or created in accordance with one element of the present invention. The article 10 may take any one of a number of forms created by inter-mixing one or more filaments 12a, 12b, 12c, 12d (for example). Intermixing may be by way of: weaving, as shown in FIG. 1; platting or knitting, as shown in FIGS. 2a to 2c; twisting/platting, as shown in FIGS. 3a to 3c, filaments 12 may be embroidered onto a material or fabric or may comprise a wadding of randomly distributed filaments (non-woven form), as shown diagrammatically in FIG. 4. Such filaments lend themselves to the manufacture of articles such as surgical sutures, artificial ligaments, tendons, devices for knee or hip implants, vascular grafts, nerve repair conduits, ties, wadding for wound dressing, protective covers, textiles for hernia repair or breast implants.

The filaments described above may be of natural materials such as silk, cotton, wool and the like or may be of man-made material such as Polyester, Polyethylene, polypropylene, polytetrafluoroethylene, prolene, nylon, polydioxanone. The surfaces of some of these materials may already be hydrophilic as is the case for silk, cotton, and some wools but man-made materials may not necessarily have a naturally hydrophilic surface and may need to be treated or modified to make them suitable for use in the present arrangement. Details of one process for making the surface of a man-made material hydrophilic is described alter herein but it will be appreciated that other ways of achieving a hydrophilic surface may be used. Some such alternatives include roughening the surface with an abrasive, acid etching, electron bombardment etc. Any such process effectively modifies the surface of the material forming the filament, such as to increase the hydrophilicity thereof.

The process of attaching bacteriophages to the filament commences with the preparation of a solution 16 containing one or more bacteriophages 18 held in suspension in a carrier fluid. The carrier fluid may comprise Phosphate buffer solution (PBS), or Infusion broths, or SM buffer, or ethanol but other carrier fluids may be used. The carrier fluid is important because this allows the suspension of bacteriophages in something that can be applied to the filament in a manner that allows better control over the application and a greater degree of assurance over the distribution of the bacteriophages than might be possible in the prior processes. Whilst there is a broad range of concentrations that one might use of bacteriophages in the suspension fluid, it has been found that a good concentration suitable for use in the present arrangement is a concentration of bacteriophage per unit solution of between 10² PFU/mL to 10¹² PFU/mL. A concentration of bacteriophages per unit solution of between 10⁵ PFU/mL to 10⁹ PFU/mL is the preferred range as this ensures sufficient bacteriophages are present but avoid overloading the solution.

Once the solution 12 has been prepared, it may be dispensed in any one of a number of ways but it is preferred that the dispensing approach is one of drop dispensing as it has been found that this approach provides a high degree of control over the delivery process and the density of bacteriophage concentration on the finished filament and final article. There is a distinction between drops and spray coating techniques, the latter of which is well known for use as a mechanism of applying bacteriophages to an article. Spray coating techniques may dispense a plurality of droplets or micro-droplets in contrast to a drop dispenser which may dispense drops individually at a known, controlled rate. Spraying techniques whilst being suitable for some surfaces such as substantially planar surfaces do have a tendency of causing undesirable waste due to overspray and can cause off-washing of the desired bacteriophages when the carrier solution drains from the surface to which it has been applied. In such an arrangement, it is difficult to ensure good and/or accurate concentrations of bacteriophages and even coating thereof over the surface to which they have been applied. Whilst this can be accepted in some applications, it is generally compensated for by the application of much greater quantities/concentrations of bacteriophages than might be desired or even economically desirable. This problem is something that one element of the present invention is aimed at solving. Drops have a size range of between 0.2 microlitres and 100 microlitres whereas droplets tend to have a size range of between 0.005 microlitres and 0.08 microlitres. In some applications of the current invention and with advances in single micro-drop dispensing technology a smaller discrete drop size may be possible as low as 0.1 microlitres is currently possible. Importantly, a drop is dispensed and dispensable by a drop dispenser one drop at a time or as a discrete drop enabling a drop to be placed at a chosen discrete location. The present invention makes good use of the properties inherent in a drop to control the application of bacteriophages in a measured, repeatable, accurate and economical manner which is in stark contrast to the approaches of the prior art and critical for quality control. These and other advantages of the present invention will present themselves in more detail later herein.

FIG. 5 is a diagrammatic representation of a drop dispensing apparatus 1 which may be used in conjunction with other aspects of the present invention to accurately dispense and deposit drops of a carrier fluid 20 having bacteriophages 18 in suspension. The apparatus may include one or more reservoirs 40 of pre-mixed bacteriophage solution 16 containing a carrier fluid 20 having a known or pre-determined concentration of bacteriophages 18 contained therein. A mixing apparatus shown schematically at 42 in FIG. 5 having a drive mechanism 44 and mixing paddle 45 may be provided in association with one or more of the one or more reservoirs 40 such as to keep the bacteriophages 18 and carrier fluid 20 circulating within the one or more reservoirs 40, thereby to ensure a more even distribution of the bacteriophages 18 within the carrier fluid 20. It will, however, be appreciated that such a mechanism is not necessary in all applications and is certainly not needed if the solution 16 is used within a short period of being placed within the reservoir 40. Also provided on one or more of the one or more reservoirs 40 is a drop dispenser 46 which may include a solenoid 48, a piezoelectric actuator 48 or other actuation device 48 acting on a flow control means 49 such as a valve 49, so as to control the timing of the drops being dispensed therefrom. A timing controller, shown schematically at 50, may be linked to each solenoid 48 so as to manage the timing and/or duration of any drops that might be dispensed. An indexing/advancing system (not shown but represented by arrow 52) may be used to cause the motion of the filament 12 under the one or more dispensers 46 such as to allow for the dispensing of drops 54 onto the filament 12 as and when required and as described in more detail later herein. FIG. 5 illustrates an arrangement having multiple reservoirs 40 each containing the same solution 16 and represented by the letter A. When the apparatus is being used to dispense the same solution 16 the separate reservoirs 40 may be replaced by a common reservoir. FIG. 6 illustrates a slight variation on the arrangement of figure which is different purely by the presence of multiple reservoirs 40 each containing different solutions 16. This form of arrangement could be used to dispense different combinations of bacteriophage solutions A, B, C, D onto the same filament 12, but at different locations thereon. The arrangements of FIGS. 5 and 6 may be used to apply different cocktails or combinations of bacteriophages 18 to different filaments or may be used to apply the same bacteriophage to multiple filaments. Still further, they may be used to apply different bacteriophages 18 to different regions R on the same filament. Indeed, the ability to space the regions R having bacteriophages 18 applied thereto from each other solves another problem as it is well known that, when combined together in one location different bacteriophages 18 or cocktails thereof can interact negatively to each other and thereby reduce rather than enhance the effectiveness of the bacteriophage solutions 16.

FIG. 7 illustrates a filament 12 which has passed through the apparatus of FIG. 5 or 6 and which shows the dispensed drop 100 having diffused laterally as shown by arrows S within the hydrophilic surface (12a) such as to disperse the solution 16 and bacteriophages 18 along a length of the filament 12. The drops duration Dd and thus volume V or the distance moved by the advancing system 52 can be controlled by the controller 56 to control the length of filament 12 saturated by each drop and the distance between each drop respectively, such as to cause the full coating of the filament or to leave gaps G between the bacteriophage impregnated portions W, X, Y, Z. Spaced apart regions R1, R2 etc of bacteriophage coated lengths may be utilised so as to reduce the amount of solution being applied whilst ensuring that there is enough room or volume available for the adsorption of the solution 16, thereby reducing or even eliminating the possibility of wastage.

It can simply be established by experimentation that for a filament 12 of a certain size, a distance S will be saturated by a volume V of bacteriophage solution 18. The indexing/filament advance system 52 of the drop dispensing apparatus 1 is configured to move the filament 12 at least the distance S between the dispensing of each drop of volume V to provide a saturated filament with no wasted bacteriophage solution. The indexing/filament advance system 52 of the drop dispensing apparatus 1 may be configured to move the filament 12 more than the distance S between the dispensing of each drop of volume V to provide a filament with gaps G between saturated regions R. The skilled person will understand that the predetermined volume V may be supplied by a plurality of drops at each discrete location. A filament 12 with gaps G between saturated regions R may be particularly useful if a plurality of solutions 18 containing different bacteriophages or different cocktails of bacteriophages are being applied to the same filament 12. The filament advance system 52 may stop the filament 12 at the point each drop is dispensed or the filament 12 may be moved continuously by the filament advance system 52 and the dispensing of the drops by the drop dispenser 46 may be indexed to the movement of the filament 12 by the filament indexing/advance system 52.

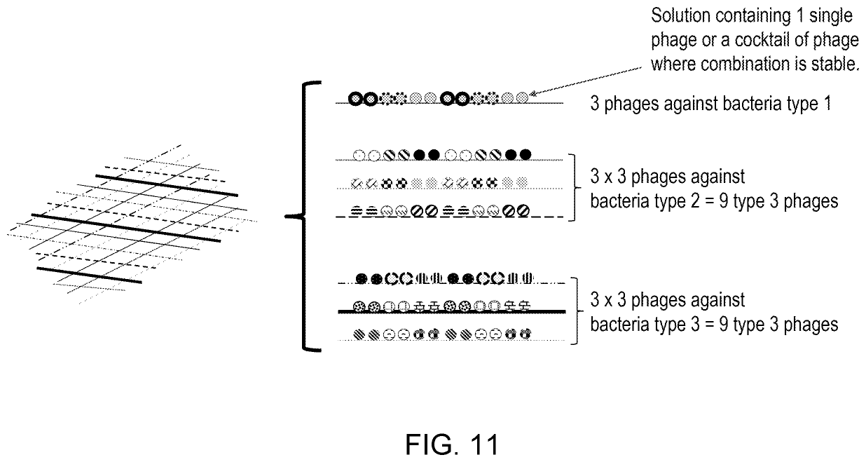

FIG. 11 shows some example configurations of combinations of bacteriophages 18 applied to a plurality of regions R1-R4 one or more filaments 12 in order to attack one or more bacteria with a plurality of phages 18 thereby providing increased efficacy.

FIG. 8 is a schematic representation of four components of the overall apparatus that could be used to pre-treat filament 12 and apply the drops 54 and also post treat the filament so as to align the bacteriophages 18 in a manner described in more detail shortly. Each component may be used individually or in combination with any one or more of the others but, preferably, they are all used together. In any arrangement, the first component comprises an optional bath 60 of, for example, Hydrogen Chloride used to clean the surface of the filament and pre-treat the surface to make it more susceptible to adsorption of subsequently applied treatments. The second, optional, component comprises a plasma generator 64 which may be surrounding the filament 12 and able to produce a plasma such as a coronal plasma for the purpose of enhancing the hydrophilicity of the surface as is well known in the art and often referred to as “surface modification” which alters the chemistry at the surface of the filament. In essence, the plasma reacts with the surface 12a of the filament 12 and makes it more porous and, hence, more susceptible to the adsorption of a fluid. This step lends itself well to use in relation to man-made fibres such as those mentioned above but is of less use in relation to natural fibres such as cotton, silk etc each of which are already relatively hydrophilic. The drop dispensing mechanism of FIGS. 5 and 6 is placed after the optional plasma generator 64 and comprises the components described above when referring to FIGS. 5 and 6 and which are not described again at this point. The final, optional, component comprises an electric field generator 66 formed of, for example, a tubular electrode 68 having a longitudinal axis X and through which the filament 12 is passed such as to be exposed to any electrical filed generated therein. An electrical potential EP is created between the filament and the electrode 68 such as to have a negative potential on the electrode and relatively positive potential on the filament. The generation of this electrical potential has the effect of causing each bacteriophage to align radially outwards of the filament in the manner shown diagrammatically in FIG. 9. The positive tails 18b of the bacteriophages 18 are attracted to the electrode 68 whilst the negatively charged heads 18a are attracted to the filament 12. This orientation remains after the filament and bacteriophages 18 pass from the electrical field generator 66 and remain in this orientation for some considerable time and/or unless re-aligned by some other external influence. This alignment is important as it is the tails of the bacteriophages which attack and destroy bacteria so orienting the bacteriophages in this manner increases the effectiveness of the bacteriophages to destroy bacterial infections adjacent the location in which they are used.

FIG. 10 illustrates a good example of a full medical product 120 which could be formed from a plurality of the filaments coated in the manner as described above. This example is an artificial ligament formed from a woven/braided riband of material 122 folded to form a soft loop end 124 and a hard loop end 126. Filaments 12 can also be delivered to a patient within a capsule 130, a pill 132 comprising filaments 12 or a fibrous capsule 134. Other forms of ligament, delivery system and indeed other forms of medical product could be made from and/or incorporating a filament 12 coated with a bacteriophage solution 16 as described above. Such medical products 120 and those shown in FIGS. 1 to 4 will comprise one or multiple filaments and each filament 12 can be provided with a plurality of regions R containing the same bacteriophage 18 or a plurality of regions R having different bacteriophages 18 before they are formed into a finished article. A finished article could have multiple Zones Z1, Z2, Z3 each of which has a different bacteriophage 18 facilitated by forming each zone from a different filament 12 where each filament 12 has a different bacteriophage 18 applied thereto or filaments 12 having bacteriophage 18 in different regions R.

The method of manufacture of the product or article 10 will now be described in flow sequence such as to more specifically exemplify elements of the process. Reference is made to the drawings in general but particularly to FIGS. 8 and 9 to describe the production of a filament 12 having bacteriophages distributed on/within the surface thereof. Firstly, a filament of material such as, for example, a man-made material is passed through an optional cleaning/etching solution in the form of, for example, Hydrogen Chloride which prepares the surface 12a of the filament 12 before subsequent steps. The next, optional step comprises the plasma treatment step conducted by passing the filament 12 through the plasma generator 64 such as to modify the surface of the filament 12 and, thereby, increase the hydrophilicity of the surface 12a. the dispensing of drops 54 of a first solution 16 having a first bacteriophage contained therein onto one or more filaments 12 either sequentially or simultaneously in a manner that dispenses a pre-determined or pre-selected amount of solution and, hence, a pre-determined or pre-selected amount of bacteriophage 18 onto one or more regions R1-R4 on the one or more filaments. The pre-determined or preselected amount of solution The step may also apply a second solution 16b containing a second bacteriophage 18b in a second carrier fluid 20b onto the same or a different filament 12 at locations spaced from each other or in close proximity to each other. Such a step effectively deposits a pre-determined or pre-selected amount of solution per unit length and, by analogy, a pre-selected or pre-determined amount of bacteriophage per unit length. This can be on one or more filaments. A gap G may be left between the deposited drops 54 such that once the drops are adsorbed or otherwise taken into the surface 12a of the filament 12 there are gaps G or separations between deposited bacteriophages or no gaps. The important thing to do is to apply just enough solution in each drop 54 as to be capable of being adsorbed by the surface 12a of the filament 12 without causing excessive amounts of waste. The drops may be dispensed at a frequency/in a quantity sufficient to saturate the entire length of the filament surface 12a or insufficient to saturate the entire length, thereby to form said defined regions R and gaps G. once the drops 54 have been deposited, they will tend to spread-out as previously discussed as they saturate the surface 12a of the filament 12. Once they have been fully adsorbed the filament 12 can be passed through a further step encompassed by a cylindrical electrode 66 and applying an electrical potential between the electrode and the filament such as to align the negatively charged heads 18a of the bacteriophages 18 against the filament surface 12a and cause the legs 18b of the bacteriophage 18 to extend outwardly from the filament 12. Once the bacteriophages are aligned the filament 12 may be would onto a former (not shown) for subsequent use or may go immediately for use in the manufacture of an article or product 10 as described above and with reference to FIGS. 1 to 4 and also FIG. 10. The method may include the steps of forming the article 10 such that it has different zones Z1, Z2, Z3 and each zone may be formed from a different filament 12 having a different bacteriophage applied thereto. Such an approach allows the matching of specific bacteriophages to different zones Z of the end article or product, and this may allow for the better matching of bacteriophages to the expected bacterial infections in different portions of the body adjacent the article or product 10.

Whilst not specifically separately claimed herein, it will be appreciated that the tubular electrode 66 and the way it is used to align the bacteriophages 18 on a filament 12 may also form a separate invention and may be claimed in future applications. It will also be appreciated that whilst the arrangement of FIG. 8 has not been claimed as an apparatus (in part or in its entirety) it may also form a separate invention and may be claimed in future applications.

For the avoidance of doubt herein discrete means single and separate from another of the same.

Any system feature as described herein may also be provided as a method feature, and vice versa. As used herein, means plus function features may be expressed alternatively in terms of their corresponding structure.

Any feature in one aspect may be applied to other aspects, in any appropriate combination. In particular, method aspects may be applied to system aspects, and vice versa. Furthermore, any, some and/or all features in one aspect can be applied to any, some and/or all features in any other aspect, in any appropriate combination.

It should also be appreciated that particular combinations of the various features described and defined in any aspects can be implemented and/or supplied and/or used independently.

### Experimental Evidence

The benefit of the invention was demonstrated by the following experiments carried out by the applicant. Filaments 12 with varying amounts and configurations of bacteriophage 18 were exposed to bacteria of varying types and the result reviewed after incubating overnight at 37° C. The efficacy of the filaments 12 was then measured.

### 1-Spacing of Drops and Effect on Antibacterial Efficacy

Groups of triplicate silk filament samples having a length of 4 cm, each received discrete drops of 4 microlitres of the same S. aureus phage solution spread along the length, with different spacings between the discrete locations at which the drops were applied. The treated filaments were compared by inhibition zone assay.

Group SA-A: all 3 samples were loaded with twelve 4 μl SAP1 drops, with minimal space between drops.

Group SA-B: all 3 samples were loaded with six 4 μl SAP1 drops, with a space equal to one drop between drops.

Group C: all 3 samples were loaded with four 4 μl SAP1 drops, with a space equal to two drop between drops.

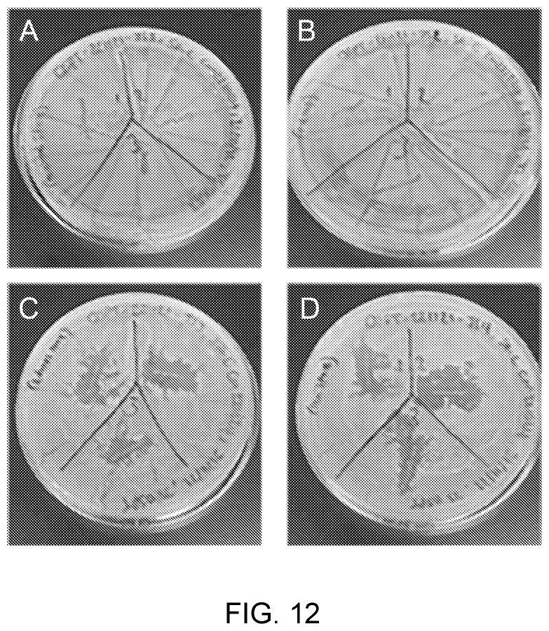

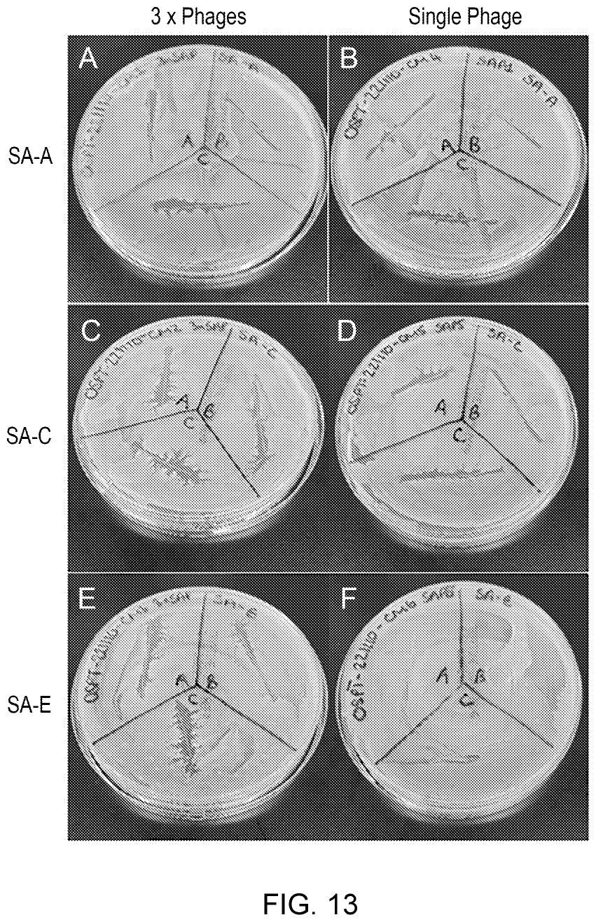

Group D: all 3 samples loaded with three 4 μl SAP1 drops, with 1 cm spaces between drops. All samples were placed onto lawns of one type of S. aureus bacteria (SA-C) and left to incubate at 37 C overnight. The efficacy of the filament 12 was assessed by measuring the area of the lysis zone which is the darker area in the images of FIGS. 12. The surface area was measured using Image-J.

| Samples | Surface area (pixels) | AVG surface area |
|---|---|---|
| A-1 | 184 | |
| A-2 | 134 | |
| A-3 | 156 | 158 |
| B-1 | 145 | |
| B-2 | 158 | |
| B-3 | 173 | 158 |
| C-1 | 1747 | |
| C-2 | 1648 | |
| C-3 | 1143 | 1512 |
| D-1 | 1860 | |
| D-2 | 2372 | |
| D-3 | 2148 | 2126 |

The results showed a lysis zone ˜10 times larger for samples with more spaced drops, clearly demonstrating the benefit of a controlled, predetermined amount of bacteriophage solution per unit length. The results also suggest possible interference between different phage deposits and benefits in being able to control very precisely the deposition of discrete drops onto the material.

### 2-Symbiotic Effect of Multi-Phages Added to Silk Filaments

A total of 6 groups of treated filaments were compared by inhibition zone assay.

For all 6 groups, triplicate silk filament samples of 4 cm in length, each received 6 discrete drops of 4 microlitres phage solution spread along the length.
Groups A, C, E received alternating patterns of 2-2-2 drops of each of the different phage solutions (SAP1, SAP5 and SAP8).
Groups B, D, F received only 1 of the phage solutions each (group B received SAP1, group D received SAP5 and group F received SAP8).

All samples of groups A&B were placed onto a lawn of a first S. aureus bacteria “SAA”. All samples of groups C&D were placed onto a lawn of a second S. aureus bacteria “SAC” and”. All samples of groups E&F were placed onto a lawn of a second S. aureus bacteria “SAE”. The plates were left to incubate at 37 C overnight before comparing the zones of lysis of each group.

Surface area measured using Image-J

| | Lysis | | | Lysis | |
|---|---|---|---|---|---|
| | zone | | | zone | |
| | surface | AVG | | surface | AVG |
| | area | Surface | Single | area | Surface |
| 3 Phages | (pixels) | area | Phages | (pixels) | area |
| SAA-A.A | 180 | | SAA-B.A | 130 | |
| SAA-A.B | 284 | | SAA-B.B | 86 | |
| SAA-A.C | 118 | 194 | SAA-B.C | 241 | 152 |
| | | ~20% larger | | | |
| SAC-C.A | 303 | | SAC-D.A | 212 | |
| SAC-C.B | 454 | | SAC-D.B | 136 | |
| SAC-C.C | 447 | 401 | SAC-D.C | 267 | 205 |
| | | ~50% larger | | | |
| SAE-E.A | 366 | | SAE-F.A | 0 | |
| SAE-E.B | 274 | | SAE-F.B | 0 | |
| SAE-E.C | 398 | 346 | SAE-F.C | 0 | 0 |

The results show that the filaments which had received an alternating pattern of all 3 phages had larger lysis zones than the filaments which had only received one of the types of phages. The discrete method of deposition therefore provides an increased antibacterial efficacy, whilst precisely controlling the amount of different phages added to the filaments. This demonstrates the possibility of adding a range of different bacteriophages 18 or cocktails of complementary bacteriophages 18 onto different filaments 12 within one same device, therefore enabling the development of devices active against multiple types of bacteria (ex: S. aureus, E. coli, Pseudomonas.a etc.,).

3-Complete Killing of Bacteria for 7 Days Following 4 Re-Exposures to S. aureus Bacteria

Triplicate short 1 cm filaments were prepared by depositing a 4 microlitre drop of S. aureus phage solution (OSPT), were compared to short 1 cm pieces of commercially available Vicryl Plus sutures (Vicryl Plus).

All samples were added to microtubes containing 200 μL of a 10′5 CFU/mL S. aureus bacteria solution and were left in an incubator at 37 C, together with a S. aureus bacteria only control (Contr.), for an initial period of time of 24 h. After 24 h, each sample was removed from its respective bacteria solution and placed in a new microtube containing 200 μL of S. aureus, left at 37 C for an additional 24 h. In the meantime, all extracts of the first 24 h microtubes were diluted 9 times and plated onto agar plates (BHIA), left overnight at 37 C. The samples were transferred to new microtubes and new bacteria solutions 3 successive times, up to a total of 168 h.

| Successive tests | CFU/mL, OSPT | | | CFU/mL Vicryl Plus | | | CFU/mL B. Contr. |
|---|---|---|---|---|---|---|---|
| T = 24 h | 0 | 0 | CG* | ** | 3.3 × 10⁶ | 1.3 × 10⁷ | 2.7 × 10⁹ |
| T = 48 h | 0 | 0 | 0 | 8.6 × 10⁸ | ** | 2.7 × 10⁹ | 2.3 × 10⁹ |
| T = 72 h | 0 | 0 | 0 | 1.0 × 10⁹ | ** | 4.3 × 10⁸ | 9.3 × 10⁸ |
| T = 168 h | 0 | 0 | 0 | ** | 1 × 10⁸ | 4.0 × 10⁶ | 3.3 × 10⁹ |

*CG: unusual localised growth at the higher dilutions but not present in the undiluted solution, suggesting accidental contamination growth rather than growth from the extract.
** Wet plate led to merged colonies which prevented the calculations of exact number.

The results showed there was no bacteria growth at all in any of the solutions which had received OSPT samples, which was in contrast to the commercial antibacterial Vicryl Plus sutures where bacteria grew to similar levels to the controls after 24 h. This demonstrates a complete elimination of the bacteria when exposed to OSPT treated filaments, using a small, controlled and predetermined volume of phage solution per unit length.

1. A method of manufacturing an article (10) comprising one or more filaments (12) of material having a hydrophilic surface (12a) comprising the steps of:
a) forming a first solution (16) containing one or more bacteriophages (18a) in suspension in a carrier fluid (20);
b) taking one or more first filaments (12); and
c) depositing upon said one or more first filaments (12) said first solution (16) containing said one or more bacteriophages (18)
characterised in that said deposition of said first solution (16) is by dispensing drops (22) thereof from a drop dispenser (24) and by the step of depositing a pre-determined amount of said first solution per unit length on said first filament (12) and
forming said one or more first filaments (12) into a final article (10).

2. A method as claimed in claim 1 characterised by the further step of forming a second solution (16b) containing one or more second bacteriophages (18b) different from said first bacteriophages (18a) in suspension in a second carrier fluid (20b); taking one or more second filaments (12b); and depositing upon said one or more second filaments (12b) said second solution (16b) containing said one or more second bacteriophages (18b) a pre-determined amount of said second solution per unit length on said second filament (12) and forming said one or more second filaments (12b) into a final article (10) along with said first filaments (12a).

3. A method as claimed in claim 1 and the one or more first filaments (12) each having a first region R1 discrete from a second region R2;
the method including the further steps of forming a second solution (16b) containing one or more second bacteriophages (18b) different from said first bacteriophages (18a) in suspension in a second carrier fluid (20b);
depositing upon said first region R1 a pre-determined amount per unit length of said first bacteriophage solution (16a); and depositing upon said second region R2 a pre-determined amount per unit length of said second bacteriophage solution (16b); and forming said one or more filaments (12) into a final article (10).

4. A method as claimed in any one of claims 1 to 3 and wherein said forming step for forming said article (10) comprises one or other of: weaving, braiding, plating, knitting, embroidering an article or forming a non-woven article.

5. A method as claimed in any one of claims 1 to 4 and including the step of mixing said bacteriophages in said solution (16) at a concentration of bacteriophages (18) per unit solution of between (10² PFU/mL to 10¹² PFU/mL).

6. A method as claimed in any one of claims 1 to 4 and including the step of mixing said bacteriophages in said solution at a concentration of bacteriophages per unit solution of between (10⁵ PFU/mL to 10⁹ PFU/mL).

7. A method as claimed in any one of claims 1 to 6 and wherein said filament (12a or 12b) has a length L and including the step of depositing said solution (16a or 16b) at discrete separated positions along the length L of said filament (12a or 12b).

8. A method as claimed in any one of claims 1 to 7 and including the step of simultaneously depositing multiple drops (30a to 30d) of said solution (16a or 16b) onto said filament (12a or 12b) at discrete separated positions along said filament (12a or 12b).

9. A method as claimed in any one of claims 1 to 8 and including the step of depositing said drops (30) on said filament (12) at a spacing sufficient to saturate the entire length L of said filament (12).

10. A method as claimed in any one of claims 1 to 8 and including the step of depositing said drops (30) on said filament (12) at a spacing insufficient to saturate the entire length L of said filament (12), thereby to form discrete lengths G of said filament (12) without bacteriophages applied thereto.

11. A method as claimed in any one of claims 1 to 10 and including the step of manufacturing an article (10) having two or more zones (Z1, Z2) and forming said article (10) of filaments (12) having different bacteriophages, thereby to form an article (10) having different bacteriophages at different zones thereof.

12. A method as claimed in any preceding claim and wherein said article (10) has an end portion (10a) and a non-end portion (10b) and method further includes forming said article (10) having a first filament (12a) having a first bacteriophage at said end portion (10a) and a second filament (12b) having a second bacteriophage at said non-end portion.

13. A method as claimed in any preceding claim and including the step of mixing said bacteriophage containing solution prior to deposition on said filament.

14. A method as claimed in any preceding claim and including the step of modifying the surface of said filament to increase the surface hydrophilicity thereof.

15. A method as claimed in claim 14 and including the step of modifying the surface of the filament by passing said filament through a plasma discharge.

16. A method as claimed in any one of the previous claims including the step of passing the filament along an electrode and applying an electric potential to align the bacteriophages relative to said filament.

17. A method as claimed in claim 16 including the step of passing the filament through a tubular electrode.

18. A method as claimed in any one of the preceding claims and wherein said first solution (16) is deposited on the filaments (12) by simultaneously dispensing a plurality of drops (22) thereof onto said filament (12) from spaced apart drop dispensers.

19. A method of manufacturing a filament (12) comprising the steps of:
a) forming a first solution (16) containing one or more bacteriophages (18a) in suspension in a carrier fluid (20);
b) taking one or more filaments (12); and
d) depositing upon said one or more first filaments (12) said first solution (16) containing said one or more bacteriophages (18),
characterised in that said deposition of said first solution (16) is by dispensing drops (22) thereof from a drop dispenser (24) and by the step of depositing a pre-determined amount of said first solution per unit length on said first filament (12).

20. A method as claimed in claim 19 and the one or more first filaments (12) each having a first region R1 discrete from a second region R2;
the method including the further steps of forming a second solution (16b) containing one or more second bacteriophages (18b) different from said first bacteriophages (18a) in suspension in a second carrier fluid (20b);
depositing upon said first region R1 a pre-determined amount per unit length of said first bacteriophage solution (16a); and depositing upon said second region R2 a pre-determined amount per unit length of said second bacteriophage solution (16b).

21. A method as claimed in claim 19 or claim 20 and including the step of mixing said bacteriophages in said solution at a concentration of bacteriophages per unit solution of between (10² PFU/mL to 10¹² PFU/mL).

22. A method as claimed in any one of claims 19 to 21 and including the step of mixing said bacteriophages in said solution at a concentration of bacteriophages per unit solution of between (10⁵ PFU/mL to 10⁹ PFU/mL).

23. A method as claimed in any one of claims 19 to 22 and wherein said filament (12a or 12b) has a length L and including the step of depositing said solution (16a or 16b) at discrete separated positions along the length L of said filament (12a or 12b).

24. A method as claimed in any one of claims 19 to 23 and including the step of simultaneously depositing multiple drops (30a to 30d) of said solution (16a or 16b) onto said filament (12a or 12b) at discrete separated positions along said filament (12a or 12b).

25. A method as claimed in any one of claims 19 to 24 and including the step of depositing said drops (30) on said filament (12) at a spacing sufficient to saturate the entire length L of said filament (12).

26. A method as claimed in any one of claims 19 to 25 and including the step of depositing said drops (30) on said filament (12) at a spacing insufficient to saturate the entire length L of said filament (12), thereby to form discrete lengths of said filament (12) without bacteriophages applied thereto.

27. A method as claimed in any one of claims 19 to 26 and including the step of mixing said bacteriophage containing solution prior to deposition on said filament.

28. A method as claimed in any one of claims 19 to 27 and including the step of modifying the surface of said filament to increase the surface hydrophilicity thereof.

29. A method as claimed in claim 28 and including the step of modifying the surface of the filament by passing said filament through a plasma discharge.

30. A method as claimed in any one of claims 19 to 29 and including the step of passing the filament along electrode and applying an electric potential to align the bacteriophages relative to said filament.

31. A method as claimed in claim 30 including the step of passing the filament through a tubular electrode.

32. A method of manufacturing according to any one of claims 1 to 31 wherein said drops have a size greater than 0.1 μL.

33. A method of manufacturing according to any one of claims 1 to 32 wherein said drops have a size range of between 0.2 μL and 100 μL.

34. An article (10) made in accordance with the method of any one of claims 19 to 33.

35. An article (10) comprising one or more filaments (12) of material having a hydrophilic surface (12a) and containing within said hydrophilic surface (12a) one or more bacteriophages.

36. An article (10) as claimed in claim 35 and having a plurality of different filaments (12) each having a different bacteriophage contained within the surface thereof.

37. An article as claimed in any one of claims 34 to 36 and wherein said one or more filaments (12) have a plurality of first portions P1 containing said bacteriophages separated from each other by a portion P2 containing no bacteriophages.

38. An article as claimed in any one of claims 34 to 37 and wherein said one or more filaments (12) contain bacteriophages along the entire length thereof.

39. An article (10) as claimed in any one of claims 34 to 38 and wherein said article (10) includes a first zone Z1 formed of a first filament (12) containing a first bacteriophage and a second zone Z2 formed of a filament containing a second bacteriophage.

40. An article (10) as claimed in claim 39 and wherein said first zone Z1 comprises an end (10a) of said article (10) and said second zone Z2 comprises a mid-portion (10b) of said article (10).

41. An article as claimed in any one of claims 34 to 40 and wherein one or more of said one or more filaments (10) comprise a natural material.

42. An article as claimed in claim 41 and wherein said one or more of said one or more filaments (12) comprises silk.

43. An article (10) as claimed in any one of claims 34 to 42 and wherein one or more of said one or more filaments (12) comprise a polymer material.

44. An article (10) as claimed in claim 43 and wherein said one or more of said one or more filaments (12) comprises a polymer material having a surface (12a) comprising a hydrophilic surface.
